(19) United States
(12) Patent Application Publication
(10) Pub. No.: US 2006/0130833 A1
(43) Pub. Date: Jun. 22, 2006

(54) Treatment of obstructive sleep apnea

(76) Inventor: Magdy Younes, Toronto (CA)

Correspondence Address: SIM & MCBURNEY, 330 UNIVERSITY AVENUE, 6TH FLOOR, TORONTO, ON, M5G 1R7, CA

(21) Appl. No.: 11/012,214

(22) Filed: Dec. 16, 2004

(51) Int. Cl. A61M 16/00; A62B 7/00

(52) U.S. Cl. 128/204.18; 128/204.23

(57) ABSTRACT

Obstructive sleep apnea and snoring are treated by administering a sedative or hypnotic drug, including barbiturates and antihistamines.

### FIELD OF INVENTION

The present invention relates to the treatment of sleep apnea and snoring.

### BACKGROUND OF THE INVENTION

The passive human upper airway (UA) is a collapsible tube with a relatively high compliance; its dimensions change substantially as a function of small changes in intra-luminal pressure. At atmospheric luminal pressure, UA cross-sectional area varies considerably among different subjects with the range extending from zero (complete closure) to >50% of maximum area. Subjects in whom the passive UA is closed, or nearly closed, at near atmospheric luminal pressure (susceptible subjects) clearly require an UA dilating force to maintain adequate inspiratory flow.

The pharynx is equipped with powerful muscles that can, when activated, counteract its tendency to collapse even under extreme negative luminal pressures. These are called the pharyngeal dilators. The most powerful of these is the tongue muscle, the genioglossus. During wakefulness UA dilator muscles (dilators) provide the necessary force to permit an adequate flow through the pharynx in all subjects regardless of how collapsible their passive UA is. This dilator activity is substantially lost at sleep onset indicating that during sleep dilators are minimally active at resting levels of respiratory drive. During sleep, however, ventilatory demand is low and the inspiratory flow required to maintain normal blood gas tensions is very modest (0.2 to 0.5 l/sec, personal observations). Subjects in whom UA can permit this level of flow without dilator activity can withstand the sleep-related inhibition of dilators. However, subjects in whom passive UA cannot permit the required flow must recruit dilators through reflex (i.e. consciousness independent) mechanisms if they are to remain asleep. Dilators can be recruited reflexly via changes in blood gas tensions and in afferent activity of mechanoreceptors that respond to negative airway pressure. However, the same chemical and mechanoreceptor stimuli result in arousal from sleep. Failure of reflex mechanisms to adequately activate the dilators will result in deterioration of blood gas tensions and progressive increase in inspiratory effort finally leading to arousal. The arousal-mediated dilator recruitment subsides when sleep resumes and the cycle repeats. This results in recurrent obstructions with oxygen desaturation and arousal from sleep, a syndrome referred to as obstructive sleep apnea (OSA). The recurrent desaturation and sleep fragmentation have been shown to result in a decrease in cognitive function and quality of life, increased risk of occupational and recreational accidents and an increased risk of high blood pressure with its attendant cardiovascular complications. It is estimated that 4 to 8% of adult males and 3 to 5% of adult females suffer from clinically significant OSA.

Snoring is another manifestation of the tendency of the human pharynx to collapse. Here, the pharynx also collapses during sleep but the degree of collapse is not so extreme as to seriously impair the exchange of oxygen (O₂) and carbon dioxide (CO₂). The patient continues to sleep but the vibrations caused by the passage of air through the narrowed floppy tube result in the noise of snoring. Snoring is extremely prevalent affecting approximately half of adult males and a quarter of adult females. Apart from its social complications, there is increasing evidence that it also may result in sleep fragmentation (the high upper airway resistance syndrome) and may also predispose to high blood pressure.

Role of arousal from sleep in the pathogenesis of obstructive sleep apnea:

When recurrent obstructive events occur during sleep, brain activity typically shows an arousal pattern at about the time the upper airway opens again. This association between arousal and upper airway opening has led to a long enduring dogma, namely that these patients are not able to compensate for the abnormal collapsibility of their pharynx unless they wake up. It is thus widely believed that arousal from sleep is a life saving mechanism in these patients and, by extension, that any effort to suppress arousals is both inappropriate and potentially dangerous. Sedatives and hypnotics are currently contraindicated in patients with obstructive sleep apnea.

I have challenged the dogma that patients need to arouse from sleep to open their airway in an extensive study published recently in two parts (Younes M. Contributions of upper airway mechanics and control mechanisms to severity of obstructive apnea. Amer J Respir Crit Care Med. 168:645-658, 2003. Younes M. Role of arousals in the pathogenesis of obstructive sleep apnea. Amer J Respir Crit Care Med. 169:623-633, 2004). In the first of these (Amer J Respir Crit Care Med. 168:645-658, 2003), I documented in detail what I, and many others, have noted for many years, namely that patients with OSA often develop lengthy periods of stable breathing during sleep. I did raise the following question: How can one conclude that arousal is necessary for compensation in a patient who shows lengthy periods of stable breathing, without arousal, at certain times during the night? There were three possible explanations: 1) Passive collapsibility of the pharynx varies from time to time, with periods of stable breathing occurring when collapsibility is less severe. 2) Transition into deep sleep (so-called delta sleep) was responsible. It is common knowledge that OSA patients usually develop stable breathing if they enter delta sleep and some investigators had shown in normal subjects that pharyngeal dilator activity tends to be higher in delta sleep. The prevailing view was, therefore, that delta sleep is inherently associated with higher dilator activity that counteracts the anatomic defect in this sleep state. 3) Patients who develop periods of stable breathing are capable of mounting effective compensation without arousal.

In the aforementioned study (Amer J Respir Crit Care Med. 168:645-658, 2003), I explored the first two possibilities. With respect to the first possibility, I found that, although passive collapsibility of the pharynx varies somewhat from time to time, the extent of this variability can in no way explain periods of stable breathing in most patients. Furthermore, I found that delta sleep does not inherently lead to increased pharyngeal stability. Thus, I concluded that when a patient displays a period of stable breathing without arousals at some point during the night the patient has demonstrated that he/she can mount effective compensation without arousal via innate reflex mechanisms.

While this conclusion clearly challenged the dogma that arousal from sleep is necessary for compensation, it raised the interesting following question: If a patient can compensate for the abnormal anatomy at some point(s) during the night, why does he/she develop recurrent obstructions with arousal during other parts of the night?

In the second component of the study (Amer J Respir Crit Care Med. 169:623-633, 2004), I suggested that premature occurrence of arousal preempts an orderly response that would otherwise be mounted by reflex mechanisms as a result of some increase in chemical drive to breathe (increased CO₂ and/or decreased O₂ level in the blood). According to this hypothesis, the premature occurrence of arousal sets the stage for recurrence of the obstruction by causing a ventilatory overshoot (overcompensation) that reduces chemical drive to breathe promoting re-obstruction upon resumption of sleep. Thus, the proposal was that arousals are not only unnecessary, but that they also are a major contributor to the severity of obstructive apnea. I further suggested that stable breathing occurs during delta sleep because of the high arousal threshold in this state, which permits chemical drive to increase to a higher level, increasing the possibility of adequately activating the dilators without arousal. A large body of evidence was presented in this study (Amer J Respir Crit Care Med. 169:623-633, 2004) in support of these hypotheses. Thus I found that:

1) The temporal relation between arousal from sleep and upper airway opening at the end of the obstructive episode does not support the contention that arousal is the mechanism by which upper airway opens. Thus, in 17% of cases, upper airway opening occurred without arousal. In a further 22% of cases, arousal occurred after the airway had opened (i.e. opening occurred prior to arousal). Furthermore, in an additional 30% of cases the occurrence of arousal did not result in opening immediately. Rather upper airway opening occurred later. This type of temporal relation strongly favoured an incidental association between arousal and opening rather than one in which arousal causes the opening. Because both arousal and arousal-independent reflex activation of dilators are triggered by the same mechanisms, the threshold for both may be reached at the approximately the same time.

2) The time of upper airway opening was the same whether arousal occurred before or after upper airway opening or did not occur at all.

3) Where upper airway opening occurred before or without arousal, the increase in air flow was more than adequate to restore a normal ventilation. Thus, arousal is not required to insure that the response is adequate.

4) When arousal occurred at the time of opening or soon after, the increase in air flow was markedly increased and was well beyond what is required. This ventilatory overshoot is expected to promote recurrence of the obstruction as pointed out above.

5) The likelihood of occurrence of a second obstruction was significantly increased when arousal occurred at the end of the first induced obstruction. This confirms the adverse effect of arousal.

### SUMMARY OF INVENTION

I have now found that, in many patients, arousal from sleep is not only unnecessary but promotes recurrence of obstruction and an increased severity. In accordance with the present invention, delaying arousals, using appropriate sedatives/hypnotics, allows the reflex mechanisms to compensate without arousal and mitigate the severity of the disorder. There are two important caveats, however:

1) It may be argued that, by delaying the arousal, blood gas tensions may have to deteriorate to unacceptable levels before reflex mechanisms succeeded in restoring airway patency. This issue was addressed in the same study (Amer J Respir Crit Care Med. 169:623-633, 2004). Thus, in this study patients were placed on Continuous Positive Airway Pressure (CPAP). Obstructive events were induced by suddenly lowering CPAP level. While on CPAP, chemical drive is perfectly normal because upper airway resistance is normal. By determining how long it takes (after induction of obstruction) to open the airway when arousal does not occur, it is possible to estimate how much CO₂ and O₂ in the blood need to deteriorate, relative to their desirable values (i.e. on CPAP), to effect opening without arousal. I found that, while in a minority of patients large changes in chemical drive are required, in the majority the changes in blood gas tensions required to open the airway are very modest. I estimated that in the average patient an increase in PCO₂ of 2 mmHg and a decrease in O₂ saturation of 3% would suffice. These changes are clinically very acceptable. Because the latency to opening without arousal and the latency to arousal when arousal occurred were, on average, similar the same findings indicated that in most patients arousal threshold is quite low; arousal occurs when only minor increases in chemical drive develop. These findings indicated that, in most patients, the arousal threshold can be safely increased.

2) By necessity, sedatives/hypnotics must be administered systemically. As such, they reach all parts of the brain and not only the areas responsible for arousal. Because sedatives/hypnotics are basically depressants to nervous tissue, the possibility exits that a sedative may suppress reflex activation of pharyngeal dilators at the same time it suppresses arousal. Such an occurrence would defeat the purpose of delaying arousal; namely to permit reflex mechanisms to activate the dilators. If suppression of reflex activation and suppression of arousal are of equal magnitude, nothing is gained and the net effect would simply be a delay in upper airway opening with worsening of the blood gas tensions. Accordingly, for a sedative/hypnotic to be effective in therapy of OSA it should suppress arousal without suppressing, or without suppressing as much, the reflex activation of dilators. In this fashion reflex activation of dilators can progress to higher level while sleep continues.

While the effect of a number of sedative/hypnotic drugs on reflex activation of dilators has been studied, my recent findings indicate that the important variable to be considered is not whether a particular drug enhances or depresses reflex activation. Rather, the important question is the relative effect of the drug on arousal and reflex activation mechanisms. A drug that depresses arousal may be suitable even if it depressed reflex activation provided the former effect is greater. On the other hand, a drug that enhances reflex activation may be unsuitable if it concurrently accelerates arousal.

In summary, my recent findings lead to two novel conclusions:

1) Selected patients with obstructive sleep apnea can be treated by sedatives/hypnotics. This notion is not only novel, it is almost heretical; these drugs are currently contraindicated in OSA patients. Suitable patients are those who a) develop arousals frequently at or near the end of obstructive events, b) demonstrate an ability to compensate in the absence of arousals by, for example, developing periods of stable breathing during sleep or where some of the obstructive events are relieved prior to the appearance of arousal and c) have a low arousal threshold as demonstrated by brief duration of obstructive events and only modest changes in O₂ saturation prior to arousal.

2) The potential effectiveness of a drug in treating OSA is measured not by its independent effect on reflex activation of airway dilators but, rather, by measuring the balance of its effects on arousability and reflex responses of dilators. This is novel in that, so far, research into drug treatment of OSA has focused on finding drugs that potentiate the activity of dilators without regard to the drugs' effects on arousability.

Accordingly, in one aspect of the present invention, there is provided a method for treating obstructive sleep apnea comprising the use of sedative or hypnotic drugs that have been shown to increase arousal threshold (the level of respiratory stimuli required to cause arousal from sleep) without depressing, or without depressing as much, the reflex activation of pharyngeal dilator muscles.

### BRIEF DESCRIPTION OF DRAWINGS

FIG. 1A is a graphical representation of the response of genioglossus activity (EMG_{gg}) to increasing PCO₂ with and without a sedative drug;

FIG. 1B is a graphical representation of the response of genioglossus activity (EMG_{gg}) to increasing PCO₂ with and without a drug that enhances reflex response of the dilator but concurrently reduces arousal threshold;

FIG. 2 shows separately the electroencephalogram (EEG), neck muscle electrical activity (EMG_{neck}), genioglossus activity (EMG_{gg}), diaphragm activity (EMG_{dia}) and chamber CO₂ concentration in experimental animals for experiments conducted herein and described below;

FIG. 3 shows the peak genioglossus activity in rats in experiments described below using pentobarbital;

FIG. 4 shows tracings of baseline activity as determined in the experiments described below;

FIG. 5 shows the peak genioglossus activity in rats in experiments described below using Phenobarbital; and

FIG. 6 shows the peak genioglossus activity in rats in experiments described below using diphenhydramine.

### GENERAL DESCRIPTION OF INVENTION

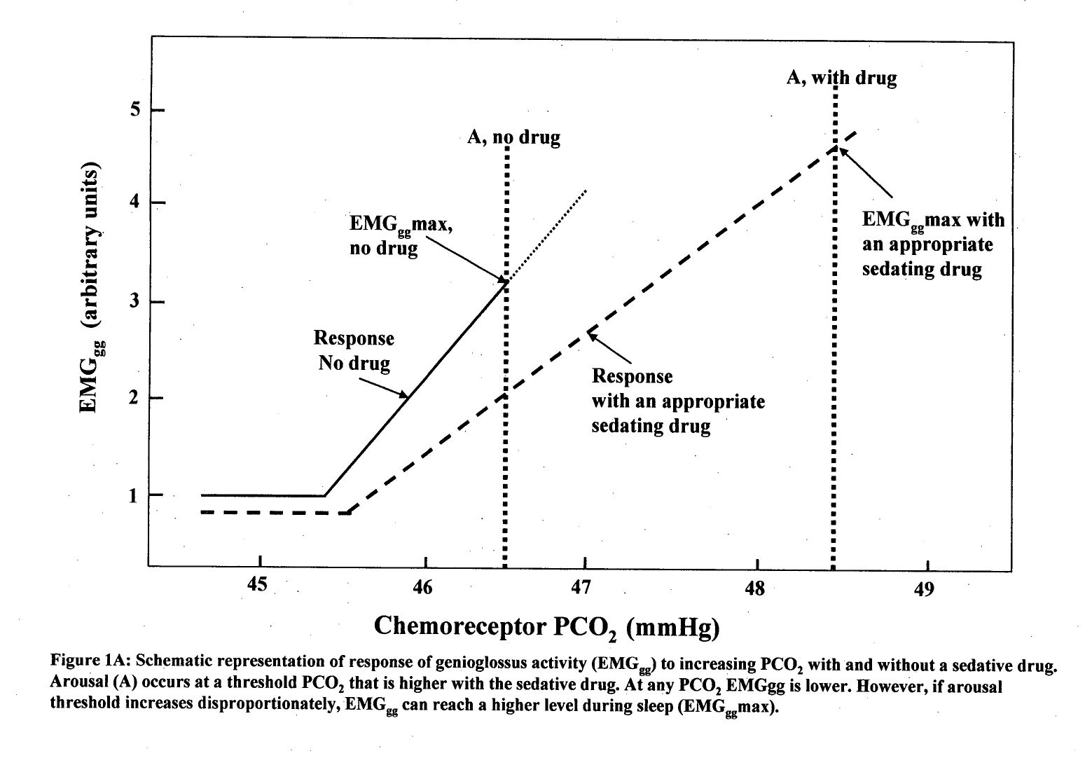

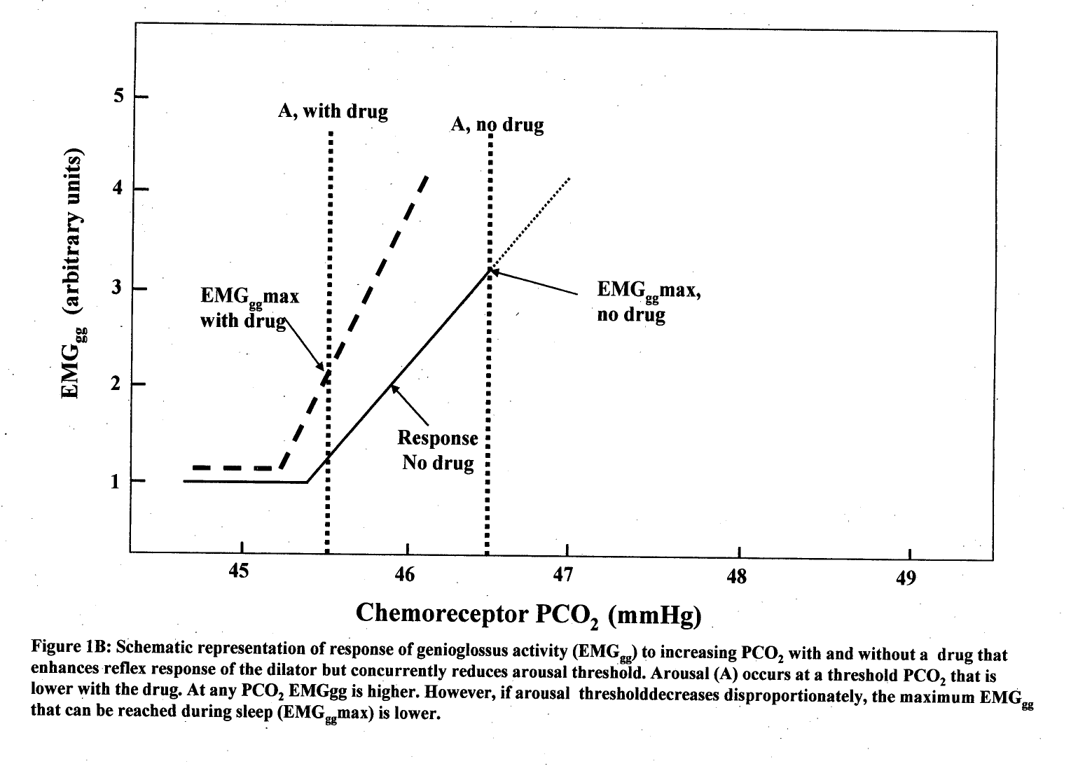

The balance between the effect of a drug on respiratory arousal threshold (the level of respiratory stimuli required to cause arousal from sleep) and on reflex response of dilators to respiratory stimuli can be assessed in several ways. One suitable approach is the one depicted in FIGS. 1A and 1B. A suitable respiratory stimulant is applied in increasing amount during sleep. The stimulant may be an increasing level of inspired CO₂ or a decreasing level of inspired O₂ concentration. Stimulus intensity is progressively increased until arousal occurs, as judged by the electroencephalogram. The activity of a suitable dilator muscle (e.g. the genioglossus) is concurrently recorded and the activity level immediately preceding arousal is recorded. This activity (EMG_{gg}max; see FIGS. 1A and 1B) represents the maximum amount of activation that can be attained during sleep by respiratory stimuli. This procedure is performed with and without the administration of a test drug. In FIG. 1A, the test drug is a sedative drug and, in FIG. 1B, the test drug is one that enhances reflex response but concurrently reduces arousal threshold. An increase in dilator activity immediately preceding arousal indicates a favourable balance between effects on arousal and on reflex activation of dilators. Such studies can be made on humans but preliminary testing in experimental animals would be prudent.

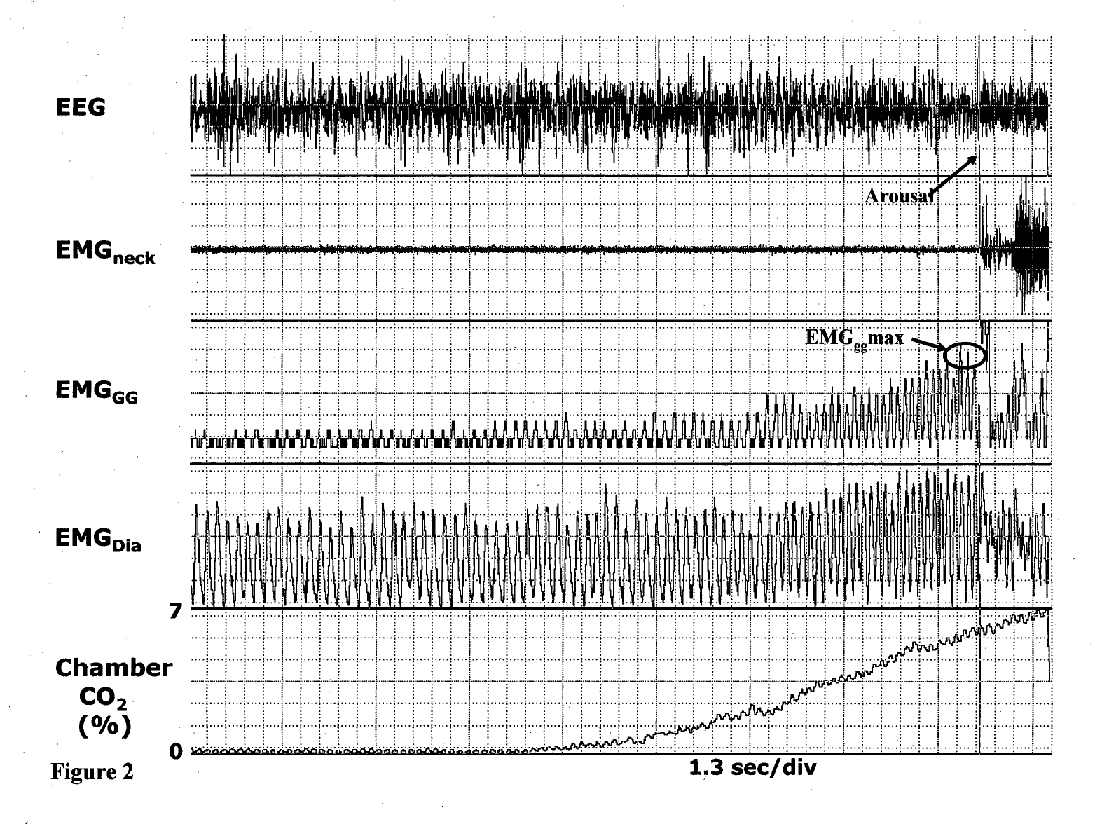

The approach just outlined (FIGS. 1A and 1B) was implemented in a freely behaving intact rat. The animal was instrumented with chronic electrodes to measure brain activity as well as activities in the genioglossus, neck muscles and diaphragm. After recovery, while the rat was naturally sleeping, the CO₂ concentration in the chamber housing the rat was increased in a ramp-like fashion until arousal was evident in the electroencephalogram. FIG. 2 shows an example of the responses obtained. This figure shows sequentially the electroencephalogram (EEG), neck muscle electrical activity (EMG_{neck}), genioglossus activity (EMG_{gg}), diaphragm activity (EMG_{dia}) and chamber CO₂ concentration. As chamber CO₂, increases there was an increase in activities of both the diaphragm and genioglossus. Arousal occurred at the arrow as evidenced by the changes in the EEG and EMG_{neck}. The maximum genioglossus activity reached just before arousal was noted (EMG_{gg}max). Such observations were made when the animals received a drug before the monitoring or received a placebo injection.

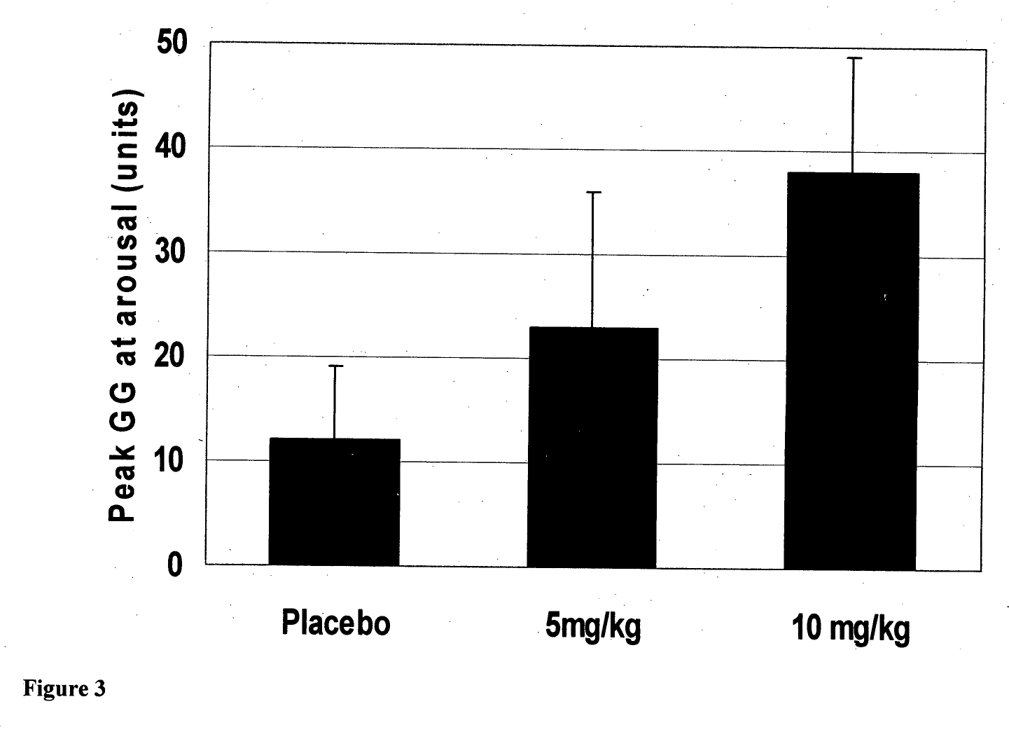

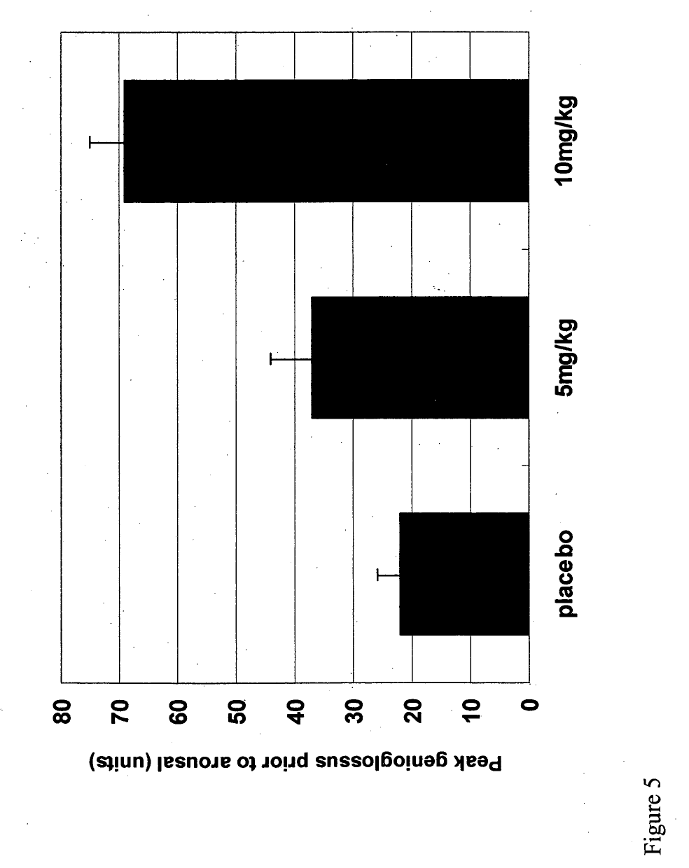

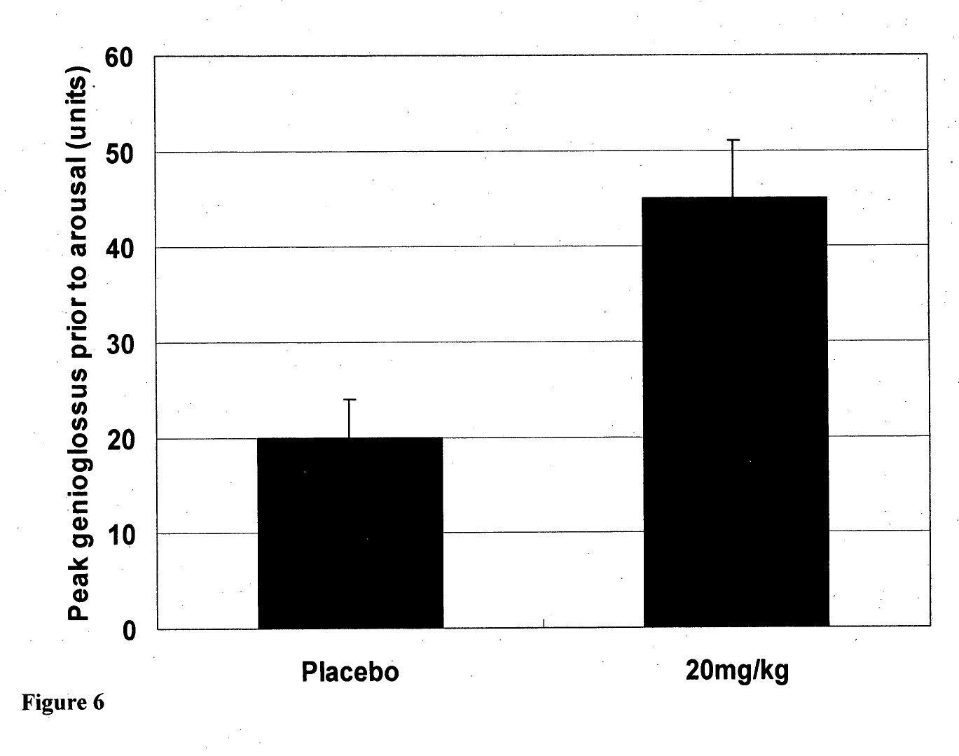

So far three drugs have been tested in the manner described above. These are pentobarbital and phenobarbital, both of the barbiturate family, and diphenhydramine, a sedating antihistamine. All three drugs gave similar results. FIG. 3, which is representative of the two other drugs as well, shows the effect of pentobarbital in mildly sedating doses in the rat (5 and 10 mg/kg). FIGS. 5 and 6 show the results with phenobarbital and diphenhydramine respectively. As can be seen, EMG_{gg}max increased in a dose-dependent fashion with drug administration, a favourable response.

Although the current findings indicate that three specific drugs meet the requirements for potential effectiveness in OSA, a person skilled in the art would recognize other members of the same families of drugs (barbiturates, antihistamines), or members of other drug families would have similar properties (favourable balance between effects on arousal and reflex activation of dilators). A person skilled in the art would also recognize that modifications to the drugs may be implemented to enhance their desired action and/or to reduce side effects. Such modifications fall within the scope of the invention.

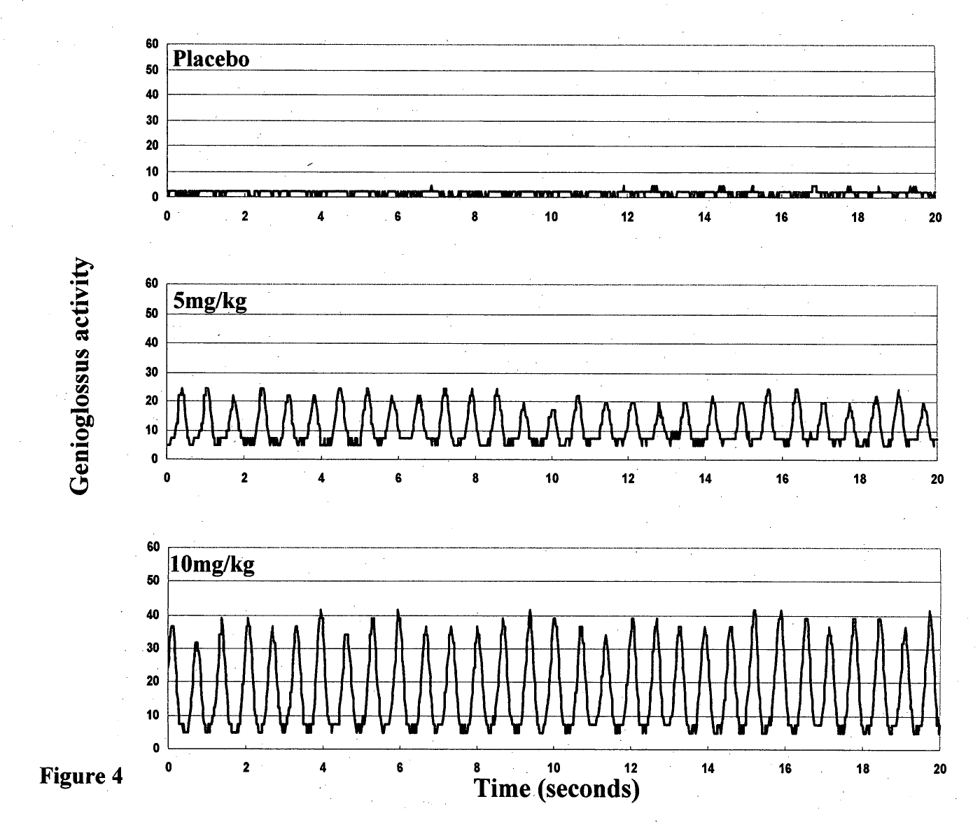

An interesting finding from this study was that not only did genioglossus activity just prior to arousal increase, but this muscle's activity was also higher during sleep while animals breathed room air, prior to delivering the CO₂ ramp. An example of baseline activity (prior to CO₂ stimulation) in one animal following placebo, 5 mg/kg and 10 mg/kg pentobarbital is shown in FIG. 4. This finding which was common to all three drugs tested, indicating that drugs in these two classes (barbiturates, antihistamines) are effective in treating snoring and not only obstructive sleep apnea. Clearly, an increase in dilator activity without an increase in chemical drive results in a more patent airway during sleep, which should mitigate snoring.

Accordingly, in a further aspect of the present invention, there is provided a method for treating snoring comprising the use of a sedative or hypnotic drug that, in addition to sedation, results in an increase in pharyngeal dilator muscle activity during room air breathing.

### SUMMARY OF DISCLOSURE

In summary of this disclosure, the present invention provides methods of treating obstructive sleep apnea and snoring by the administration of certain drugs. Modifications are possible within the scope of this invention.

What I claim is:

1. A method of treating obstructive sleep apnea in a human, which comprises:
administering to the human an effective amount of at least one sedative and/or hypnotic drug, said sedative and/or hypnotic drug being effective to increase arousal threshold without depressing or without depressing as such, the reflex activation of pharyngeal dilator muscles.

2. A method of treating snoring in a human, which comprises administering to the human an effective amount of at least one sedative and/or hypnotic drug, said sedative and/or hypnotic drug being effective to increase pharyngeal dilator muscle activity during room air breathing.
